(19) United States
(12) Patent Application Publication
(10) Pub. No.: US 2003/0082211 A1
(43) Pub. Date: May 1, 2003

(54) Proteins for use as carriers in conjugate vaccines

(76) Inventors: Andrew Richard Gorringe, Salisbury (GB); Andrew Robinson, Salisbury (GB)

Correspondence Address: Ranjana Kadle, Hodgson Russ, Suite 2000, One M & T Plaza, Buffalo, NY, 14203, US

(21) Appl. No.: 10/221,541

(22) PCT Filed: Mar. 27, 2001
(86) PCT No.: PCT/GB01/01361

(51) Int. Cl. A61K 39/02; C08B 37/00; C07K 14/195

(52) U.S. Cl. 424/234.1; 530/395; 536/53

(57) ABSTRACT

Vaccine conjugates are described comprising a capsular polysaccharide antigen conjugated to a carrier molecule, which carrier molecule is a protein associated with iron uptake by pathogenic microorganisms. The vaccine conjugate may comprise second or additional antigens also conjugated to the carrier. In a preferred embodiment, the carrier is a transferrin binding protein, in particular TbpA or TbpB. Also described are methods for conjugating the antigen/s to the carrier molecule, and to the use of transferrin binding protein in the manufacture of a carrier-antigen conjugate for vaccination. Affinity matrices comprising immobilised ligand for an iron uptake protein, and the use thereof for purifying a vaccine conjugate according to the present invention are also described.

The present invention relates to proteins for use as carriers in conjugate vaccines and to preparations and purification of such conjugates.

Many pathogenic bacteria which cause severe invasive disease possess a carbohydrate capsule which is an essential virulence component. The carbohydrate capsules are potential vaccine components since antibodies directed against them are usually protective by virtue of their complement-mediated bactericidal activity. Antibodies raised against the carbohydrate are specific for the particular serogroup from which the carbohydrate was obtained; there is one major pathogenic serotype for Haemophilus influenzae, three major serogroups for Neisseria meningitidis and over 80 serotypes for a Streptococcus pneumoniae.

A major disadvantage of capsular vaccines is that carbohydrates are T-cell independent antigens and hence the immune response they elicit is low (especially in infants), short-lived, unboostable and has an affinity which does not mature. The antigens can be converted to T-cell dependency by conjugation to proteins which enhances the immune response, including providing a memory response.

Immunisation with Haemophilus influenzae type b (Hib) capsular polysaccharide protein conjugate vaccine has been demonstrated to confer protection against Hib disease in young children, and meningococcal C conjugate vaccines have recently been introduced into the UK immunisation schedules. This is a persuasive argument in favour of introducing similar paediatric vaccine strategies for the control of childhood infections with, for example, Neisseria meningitidis and Streptococcus pneumoniae. However, these new vaccines will be complex mixtures of antigens. Adverse antigenic interactions and limitations of formulation technologies were encountered during the introduction of Hib vaccination. It seems these problems will only be exacerbated when the new vaccines are included in the established paediatric immunisation programme.

The problems stem from the fact that infant immunity to polysaccharides will only operate through a T-cell dependent mechanism and, therefore, this type of vaccine requires conjugation to a carrier protein. At present the main carrier proteins available for human use are tetanus toxoid (TT) or a genetically or chemically toxoided diphtheria toxin (DT). Increased use of these carriers may not be possible as there is evidence that pre-existing immunity to them, which may arise either through maternal antibodies passively transferred in early life or immunological memory to existing vaccines, can adversely effect the immune responses to the carbohydrate moiety. Clearly such interactions may reduce the effectiveness of current polysaccharide conjugate vaccines for primary immunisation and booster applications respectively.

There is therefore the problem that extended use of the existing toxoid carriers will result in diphtheria/tetanus toxoid overload and reduced immune response to the carbohydrate conjugated to the toxoid. In addition, the toxoids require detoxification which can alter their immunological properties. Outer membrane proteins, which have been proposed as carriers, do not have the above problems, but are complex mixtures which are difficult to characterise and vary in composition from one mixture to the next.

Another difficulty relates to the practicalities of increasing the number and complexity of vaccines for paediatric immunisation. Vaccine manufacturers have been successful in producing combinations of paediatric vaccines which can be delivered simultaneously from one syringe, thus simplifying immunisation programmes. It will now be increasingly difficult technically to extend the range of components in combination vaccines using current formulation and adjuvant technologies. The prospect of re-introducing multiple injections with all of the corresponding problems of increasingly complex vaccination programmes is likely to occur unless suitable alternative delivery systems (e.g. to mucosal surfaces) are introduced.

It is therefore generally recognised that alternative carrier proteins and alternative delivery routes are required for the introduction of novel or second generation conjugate vaccines. A new carrier for antigen delivery, including polysaccharides, has been described in WO 98/58668, using B. pertussis fimbriae. Nevertheless, there exists a need for further and alternative carrier proteins for vaccine delivery.

Chhibber, S. (1995) Vaccine, vol 13, No. 2, pp. 179-184 describes vaccines comprising an iron-regulated cell surface protein conjugated to a polysaccharide, both components of the conjugate having been derived from Klebsiella pneumoniae. The polysaccharide component was obtained by mild acid hydrolysis of a lipopolysaccharide surface-exposed antigen, and the iron-regulated cell surface protein was obtained by standard extraction procedures performed on K. pneumoniae cells cultured under iron depleted conditions. The separate components were then chemically coupled by use of cyanogen bromide.

WO87/02678 describes the major iron-regulated protein of a pathogenic species of the genus Neisseria. This protein may be employed alone as a gonococcal vaccine. Alternatively, the iron-regulated protein may be conjugated to a poorly immunogenic peptide to form a further gonococcal vaccine.

WO00/25811 describes multicomponent meningococcal vaccines comprising one or more transferrin binding proteins, including two or more transferrin binding proteins conjugated together. WO00/25811 has a publication date of 11 May 2000.

EP-A-733708 describes vaccines against porcine pleuropneumonia. In more detail, transferrin binding protein from Actinobacillus pleuropneumoniae is employed in said vaccines and as diagnostic reagents.

It is an object of the invention to provide a conjugate vaccine for presentation of an otherwise poorly immunogenic carbohydrate in a conjugate which can be used in parallel with or subsequently to existing toxoid based vaccines with amelioration or reduction of the problems and potential problems hitherto encountered. A further object is to provide a carrier protein that is an alternative to the existing toxoid carriers, for manufacture of a conjugate vaccine. A still further object is to provide vaccines that can be used for vaccination against more than one pathogen in a single vaccine formulation.

The invention provides, in a first aspect novel conjugates for vaccination based upon use of iron uptake proteins as carriers, and accordingly the invention provides a conjugate comprising:

(a) a capsular polysaccharide antigen, conjugated to

(b) a carrier, wherein the carrier is a protein associated with iron uptake by pathogenic microorganisms.

Capsular polysaccharide is a term in the art as evidenced by Singleton P. and Sainsbury D. (1996). “Dictionary of Microbiology and Molecular Biology”. Second Edition, John Wiley & Sons, Chichester. In more detail, capsular polysaccharide refers to a layer of polysaccharide external to but contiguous with the cell wall of a microorganism. Capsules generally fall within one of three categories:

(i) macrocapsules or “true” capsules which are sufficiently thick to be easily visible (with negative staining) by light microscopy;

(ii) microcapsules which cannot be observed by light microscopy, but their presence may be revealed by electron microscopy or by serological techniques; and

(iii) slime layers which are diffuse secretions and which may adhere loosely to the cell surface of a microorganism. Slime layers commonly diffuse into culture medium when la microorganism is grown in liquid culture. Such layers are often too permeable for staining techniques and, as a result, are often invisible to microscopy.

Capsular polysaccharides are distinct from lipopolysaccharides and the polysaccharides derived therefrom. In more detail, the term lipopolysaccharide is commonly used to specifically refer to the endotoxic component of the outer membrane in Gram negative bacteria. Outer membrane lipopolysaccharide is a complex molecule consisting of three covalently linked regions, namely lipidA-core oligosaccharide-O-specific chain. Thus, the term lipopolysaccharide describes a complex molecule that is integral to the outer membrane of a Gram negative bacteria. In contrast, capsular polysaccharide is external to the cell membrane of a microorganism.

By way of example, the following microorganisms are capable of producing capsular or capsule-like polysaccharides: Neisseria meningitidis; Streptococcus pneumoniae; Streptococcus pyogenes; “Streptococcus milleri” group; Staphylococcus aureus; Staphylococcus epidermidis; Haemophilus influenzae; Escherichia coli; Klebsiella pneumoniae; Actinobacillus pleuropneumoniae; Pasteurella multocida; Pseudomonas aeruginosa; Moraxella catarrhalis; Mycobacterium tuberculosis; Candida albicans; Cryptococcus neoformans; and Histoplasma capsulatum.

Bacterial iron uptake proteins are particularly suitable and examples of such proteins are given in Griffiths E and Williams P (1999), The iron uptake systems of pathogenic bacteria, fungi and protozoa, “Iron and Infection: Molecular, physiological and clinical aspects, 2nd Edition” edited by J J Bullen and E Griffiths, John Wiley & Sons, Chichester, UK. Reference throughout this specification to iron uptake proteins is intended to embrace proteins involved in siderophore uptake.

The carrier proteins of the invention provide new and useful carriers for incorporation into conjugate vaccines, with the benefit of avoidance or at least amelioration of the problems and disadvantages identified in the currently existing proteins available for this purpose. Iron uptake proteins also have advantages for carrier use as described herein as they have a function during infection by the bacteria that is important for virulence, hence where there is an antibody response to these proteins it is likely to be protective.

The carrier proteins of the present invention are preferably either TonB-dependent outer membrane proteins: having at least 10%, preferably at least 20% sequence homology with meningococcal transferrin binding protein A; or having at least 10%, preferably at least 20% sequence homology with meningococcal transferrin binding protein B.

By way of example, Table 1 illustrates the homology between a number of suitable TonB-dependent outer membrane proteins and meningococcal transferrin binding protein A (TbpA).

TABLE 1
Examples of TonB-dependent outer membrane receptors and their
homology to meningococcal TbpA.

| | | | | % |
|---|---|---|---|---|
| | | | | Homology |
| | Accession | | | to K454 |
| Name | No. | Ligand | Organism | TbpA |
| TbpA | O53136 | Transferrin | N. meningitidis | 91.9 |
| TbpA | O85048 | Transferrin | M. cattarhalis | 70.0 |
| LbpA | Q9JYK5 | Lactoferrin | N. meningitidis | 63.9 |
| HgbA | Q9L6A7 | Haemoglobin | Pasteurella | 40.6 |
| | | | multocida | |
| HgbA | O86244 | Haemoglobin | Haemophilus | 40.5 |
| | | | influenzae | |
| — | P44809 | Haemoglobin | Haemophilus | 36.2 |
| | | | influenzae | |
| PfeA | Q05098 | Enterobactin | Pseudomonas | 20.6 |
| | | | aeruginosa | |
| IrgA | P27772 | Vibriobactin | Vibrio cholerae | 24.2 |
| CfrA | O07651 | Ferric siderophore | E. coli | 22.6 |
| CirA | AAG02210 | Ferric siderophore | Pseudomonas | 24.5 |
| | | | aeruginosa | |
| PfhR | Q9X5P3 | Heme | Pseudomonas | 31.1 |
| | | | fluorescens | |
| Fct(?) | Q47162 | Chrysobactin | Erwinia | 19.7 |
| | | | chrysanthemi | |
| FegA | P70921 | Hydroxymate | Bradyrhizobium | 16.9 |
| | | | japonicum | |
| PhuA | O68881 | Hemin | Pseudomonas | 25.0 |
| | | | aeruginosa | |
| PupA | P25184 | Pseudobactin | Pseudomonas | 17.1 |
| | | | putida | |
| — | Q95CO4 | Ferric siderophore | Pseudomonas | 20.5 |
| | | | spp. | |

Further examples of suitable iron uptake proteins and siderophore uptake proteins from bacteria that may be useful as carrier proteins in conjugate vaccines are illustrated in Table 2.

TABLE 2

| | Protein | Organism |
|---|---|---|
| Proteins involved in | FoxB | Escherichia coli |
| siderophore uptake | Cir | ″ |
| | lutA | ″ |
| | FhuE | ″ |
| | FhuA | ″ |
| | FecA | ″ |
| | FepA | ″ |
| | Fiu | ″ |
| | FatA | Vibrio anguillarum |
| | IrgA | Vibrio cholerae |
| | HutA | ″ |
| | ViuA | ″ |
| | PupB | Pseudomonas putida |
| | FpvA | Pseudomonas aeruginosa |
| Proteins involved in iron | TbpA | Neisseria meningitidis |
| uptake from transferrin | TbpB* | Neisseria gonorrhoeae |
| | | Haemophilus spp. |
| | | Actinobacillus |
| | | pleuropneumoniae |
| | | Pasteurella spp. |
| | | Moraxella catarrhalis |
| Proteins involved in iron | LbpA | Neisseria meningitidis |
| uptake from lactoferrin | LbpB* | Neisseria gonorrhoeae |
| | | Haemophilus spp. |
| | | Actinobacillus |
| | | pleuropneumoniae |
| | | Pasteurella spp. |
| | | Moraxella catarrhalis |
| Proteins involved in iron | HPUA* | Neisseria species |
| uptake from haem and | HpuB | ″ |
| haemoproteins | HmbR | Neisseria meningitidis |
| | HhuA | Haemophilus influenzae |
| | HgpA | ″ |
| | HemR | Vibrio cholerae |

All the above receptor proteins are potentially TonB-dependent outer membrane porins with homology to TbpA, except those marked * which have potential homology with TbpB

Preferred iron uptake proteins are those whose expression is increased or upregulated during infection—typically the expression of these proteins is found to be minimal or absent during in vitro culture in media containing iron but is increased significantly in vivo during infection by the bacteria. Thus, the carrier proteins of the invention represent antigens that are a target for antibodies during infection. By reference to iron uptake proteins herein it is also intended to refer to fragments, derivatives and variants thereof that retain at least a portion of the function of the intact protein. More preferably, antibodies raised against the fragments, variant or derivative also bind the intact protein. Further preferred iron uptake proteins of the invention either (i) bind to iron, or (ii) bind to a human protein that binds to iron, or (iii) a siderophore.

Suitable examples of iron uptake proteins that have use as carriers in vaccine conjugates are described in the review by Griffiths and Williams (1999) and include transferrin binding proteins A and B (TbpA and TbpB; Boulton et al., 1998), lactoferrin binding proteins A and B (LbpA and LbpB; Schryvers et al., 1998), major iron regulated protein FrpB (Pettersson et al., 1995), ferric binding protein A (FbpA; Berish et al., 1992) and haemoglobin receptors (HpuA, HpuB and HmbR; Lewis et al., 1999). Transferrin binding proteins may be isolated from most bacteria within the Neisseria Haemophilus Pasteurella group of organisms. Enterobacteria (Escherichia coli, etc) produce a large range of suitable proteins including the siderophore receptors FepA (Wachi et al., 1999), FhuE (Wachi et al., 1999), FhuA (Boulanger et al., 1996), lutA (Bouchet et al., 1994).

Transferrin binding proteins are an example of a protein expressed by meningococci during infection, and in particular embodiments, a conjugate of the invention is provided comprising:

(a) a capsular polysaccharide antigen, conjugated to

(b) a carrier selected from

(i) TbpA,

(ii) TbpB,

(iii) a fragment of (i) or (ii),

(iv) a derivative of (i) or (ii) and

(v) mixtures of any of (i) to (iv).

The antigen is suitably an antigenic component of a pathogenic bacteria or virus, in which context “antigen” is to be understood to encompass variants, derivatives and fragments of an antigenic component of a pathogenic bacteria or virus such that immunisation with the antigen results in protective immunity against that pathogenic organism.

Conjugation of antigen to carrier is achieved by conventional means. In an embodiment of the invention, the carrier is first derivatised and then added to a solution of antigen. It is also an option for the antigen first to be derivatised and then added to Tbp carrier, and this can be of advantage when the antigen is liable to be damaged by the derivatisation conditions, which typically include variations in pH. Typically, for conjugation of antigen to Tbp, a bifunctional group is introduced so as to link the two together.

In use of the invention, an animal is immunised with a vaccine comprising the immunogenic conjugate and is protected against challenge by the pathogenic organism from which the antigen component of the conjugate has been derived. In this sense, protection is acknowledged by survival against a challenge with a lethal dose of the pathogenic organism, or by extended life expectancy in response to challenge with such a lethal dose. Protection is also acknowledged by a patient being less affected, less ill, following challenge by a dose of pathogenic organism.

The invention is of advantage in that it provides an alternative carrier molecule for preparation of immunogenic conjugates for presentation of an antigen in combination with a T-cell epitope. The immune response to the immunogenic conjugate of the invention is enhanced compared with the immune response against isolated antigen, thus improving the efficiency compared with vaccination by antigen alone. The invention also provides an alternative to the existing toxoid carriers, and therefore overcomes the problem of toxoid overload which can occur with extended and repeated uses of vaccines containing these toxoids.

A further advantage of using iron uptake proteins as a component of the conjugate is that they can readily be obtained without contamination by toxin and do not require detoxification prior to incorporation into a vaccine. Detoxification, which is required for the diphtheria and tetanus toxins used in the art, can alter the immunological properties of the protein. A still further advantage of neisserial-derived proteins, such as Tbps, is that they also confer or enhance projective immunity against meningococcal and/or gonococcal disease, the former a paediatrically relevant pathogen, and thus a conjugate comprising Tbp induces a dual immune response.

The invention relates also to use of the immunogenic conjugate of the invention, and thus the invention also provides use of the conjugate of the invention in manufacture of a medicament for vaccination of humans or animals against a pathogenic organism from which the antigen is derived or obtained. The invention also provides a method of vaccination of humans or animals comprising administration to the human or animal of an effective immunising amount of the conjugate of the invention.

Vaccines incorporating the immunogenic conjugate of the invention can be formulated according to techniques that are standard in this art, and the vaccines can comprise conventional pharmaceutically acceptable carriers and excipients with which the skilled person will be familiar.

The conjugate of the invention, as mentioned, comprises an principal antigen which is a capsular polysaccharide. The conjugate may optionally comprise a second antigen, and reference to a second antigen embraces two or more antigens.

The source or nature of the principal or second antigen is not limited to any particular sub-group of antigens, and indeed it is possible that the principal or second antigen in isolation is not immunogenic, but only become immunogenic which incorporated into the conjugate of the invention.

Suitable second antigens include carbohydrates, polysaccharides, monosaccharides, oligosaccharides, proteins, peptides, glycopeptides, lipopolysaccharides and similar and related molecules.

Typically, the principal or second antigen will be, or will be derived from, a component of a bacterium or virus or eukaryote which appears on an outer surface of the bacteria or virus, such as a component of the bacterial cell wall, or a component of a fimbria or flagella, or a component of the outer envelope of a virus, a specific example of which is the surface antigen of hepatitis B virus. By way of illustration, the antigen can be a component of or derived from Bordetella bronchiseptica, Clostridium tetani, Cytomegalovirus, Dengue virus, Epstein-Barr virus, Flavivirus, Hepatitis A, B, C, D or E virus, Herpes Simplex virus, Influenza virus, JEV, Measles virus, Mumps virus, Mycobacterium tuberculosis, Rotavirus, Rubella virus, TBE, Vibrio cholerae, Haemophilus influenzae, Neisseria meningitidis, Streptococcus pneumoniae, Staphylococcus aureus, B.parapertussis, HIV, HPV, polio virus, Brucella, Y.pestis, Helicobacter pylori, B. burgdorferii, malaria, Candida albicans, and RSV though the invention is not to be construed as limited just to this sub-group of antigens.

In an embodiment of the invention, the antigenic conjugate comprises a carrier of the invention, such as TbpB, conjugated to two different antigens. The conjugate is thus of use in conferring or enhancing protective immunity against meningococcal disease, and also against possibly two different pathogenic organisms from which the different antigens conjugated to the carrier are obtained or derived. An immunogenic conjugate of the invention thus optionally comprises a TbpB to which, say, pneumococcal C polysaccharide and Hib capsular carbohydrate have both been conjugated. This embodiment of the invention can therefore be used to confer protective immunity against three pathogenic organisms. An advantage of this embodiment of the invention is that multiple immunities can be conferred via a single vaccine component, avoiding the need to prepare mixtures of individual vaccines and reducing the need for repeated and complex vaccination schedules using vaccines conferring immunity against just a single organism.

The conjugates of the invention are suitable for incorporation into microparticles for delivery via a large variety of routes including oral delivery. The preparation of such microparticles is described in EP-A-0266119, EP-A-0333523 and EP-A-0706792, the contents of which are incorporated herein by reference.

A particular conjugate comprises:

(a-1) a first capsular polysaccharide antigen; and

(a-2) a second antigen, different from the first; both conjugated to (b) the carrier.

In a specific embodiment of the invention, the carrier is TbpA or TbpB or a fragment or derivative thereof and the first antigen is a Hib PRP polysaccharide.

Also provided in accordance with the invention is a vaccine comprising a conjugate as described above and, further, a method of conjugating an antigen to a carrier, comprising:

(a) derivatizing a carrier, wherein the carrier is a protein associated with iron uptake by pathogenic microorganisms, and

(b) combining the derivatized carrier from (a) with the capsular polysaccharide antigen so as to allow conjugation of the antigen to the carrier.

Preferably the method comprises, in step (a), dialysing the derivatized carrier and concentrating the dialyzed, derivatized carrier, and, in step (b), separating conjugate from unconjugated antigen.

Another aspect of the invention provides use of a protein associated with iron uptake by pathogenic microorganisms, and whose expression is unregulated during infection in manufacture of a carrier-antigen conjugate for vaccination. The invention also provides a method of vaccination comprising administering an effective amount of a conjugate of the invention.

In a still further aspect, the invention provides a method of producing recombinant human transferrin comprising:

A. obtaining a clone of human transferrin, or a fragment or derivative thereof;

B. inserting said clone, or fragment or derivative thereof, into a suitable expression vector;

C. expressing the vector of (ii) in a suitable host organism; and

D. isolating the expressed gene product from said host organism.

The clone can be isolated via a PCR based method and the expression vector can be selected from the group consisting of pMTL; and pET. The host organism is a bacterium, suitably E. coli, and specific embodiments of the invention use a host selected from the group consisting of Novablue DE3; HMS 174 DE3; BL21 DE3; JM 109; RV 308; and XL1 Blue.

An alternative method is described for the production of recombinant TbpB using denaturing conditions (2M guanidine hydrochloride, 0.5% Sarkosyl) by Lissolo, L. G., et al (1995). “Evaluation of transferrin-binding protein 2 within the transferrin-binding protein complex as a potential antigen for future meningococcal vaccines”. Infection and Immunity. 63:884-90.

Once a conjugate has been obtained it is preferred to subject the crude product to one or more purification processes. It has been found particularly convenient to purify the conjugate by affinity chromatography, and preferably using an affinity matrix to which a ligand for the iron uptake protein is bound.

Hence, an affinity matrix of the invention suitably comprises immobilized ligand for an iron uptake protein, crude conjugate can be eluted through the matrix and the ligand retains the conjugate on the matrix, which conjugate can subsequently be released by conventional techniques, such as altering the pH or altering the ionic concentration. The choice of ligand corresponds to the iron uptake protein and examples of ligands include transferrin, lactoferrin, iron, haemoglobin and bacterial siderophores.

A further aspect of the invention, described in more detail below, relates to recombinant transferrin (including fragments and derivatives thereof), and the affinity matrix preferably comprises recombinant transferrin (for example, recombinant human transferrin).

An additional aspect of the invention provides a method of purifying the conjugates described, comprising eluting said conjugate through an affinity matrix comprising immobilized ligand for iron uptake protein. A benefit of this method is that the affinity matrix will only bind functional protein, as only functional protein will bind to the immobilised ligand. Thus, the eluate is both purified in respect of non-conjugate type material and is also purified in that iron uptake protein which is dysfunctional, mutated or otherwise does not bind the ligand passes through the matrix. A considerable improvement in preparation of conjugates is thus made possible as a problem in vaccines based upon polysaccharide antigens is contamination by unconjugated antigen, which can adversely affect performance of the vaccine. This problem is overcome using an affinity matrix as described. It is preferred that human transferrin be immobilised, more preferably recombinant human transferrin for purification of Tbp-containing conjugates.

The invention is now described in specific embodiments, illustrated by the accompanying drawings in which:

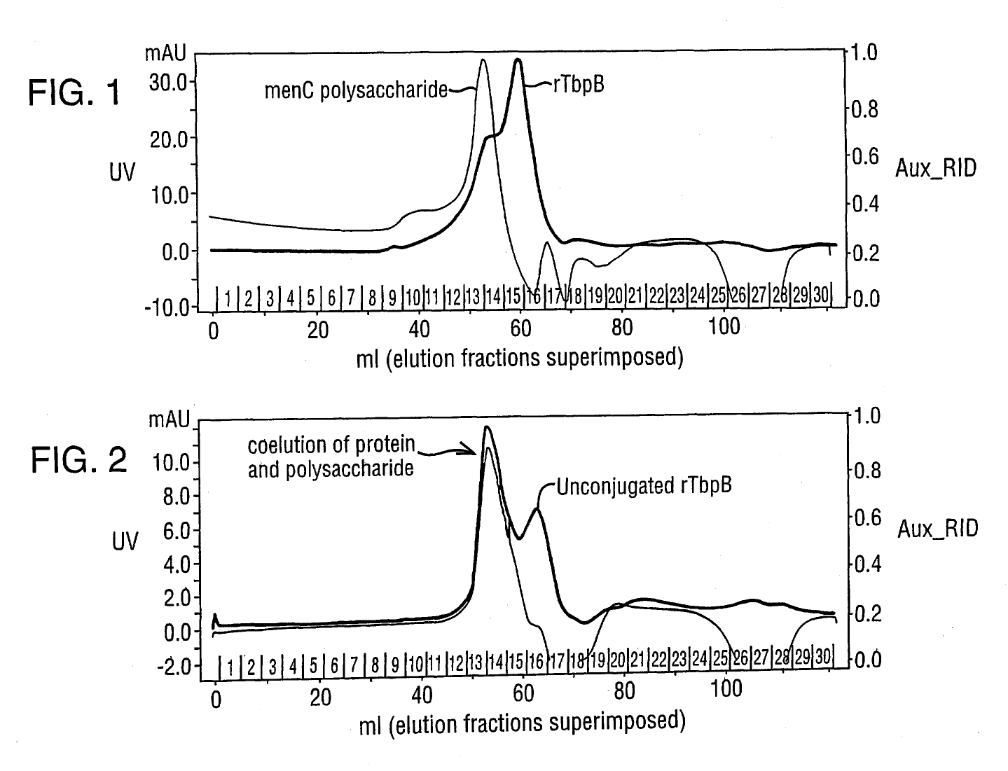

FIG. 1 shows size exclusion chromatography of an unconjugated mixture of rTbpB and menC polysaccharide;

FIG. 2 shows size exclusion chromatography of conjugated rTbpB and MenC polysaccharide;

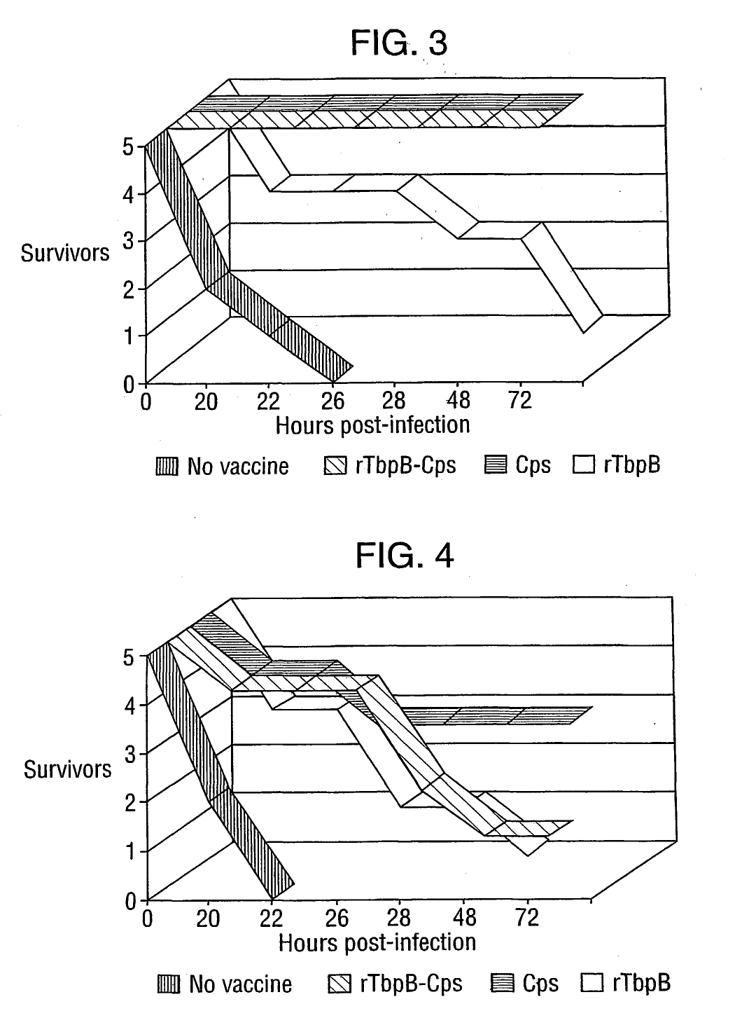

FIG. 3 shows mice protection data following administration of a conjugate according to the present invention, and subsequent challenge with 3×10⁶ dose of a serogroup C-meningococcal strain;

FIG. 4 shows mice protection data following administration of a conjugate according to the present invention, and subsequent challenge with 2×10⁸ dose of a serogroup C-meningococcal strain;

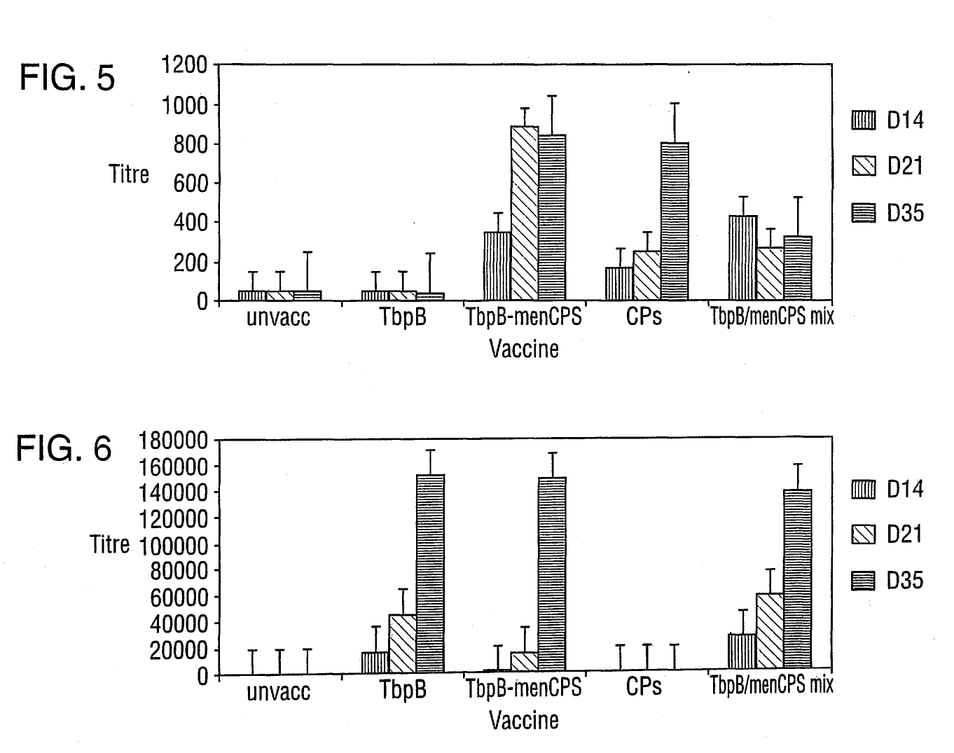

FIG. 5 shows meningococcal capsular polysaccharide-recognising antibody generation in mice following vaccination with a transferrin binding protein B-meningococcal capsular polysaccharide antigen conjugate according to the present invention; and

FIG. 6 shows transferrin binding protein B-recognising antibody generation in mice following vaccination with a transferrin binding protein B-meningococcal capsular polysaccharide antigen conjugate.

### EXAMPLE 1

### Preparation of rTbpB-Polysaccharide Conjugates

Reagents

50 mM Hepes buffer, pH 8.5

Lysine (2 mg/ml) made up in 5 ml Hepes buffer

Dimethylsuberimidate (25 mg/ml) made up in 2 ml Hepes buffer immediately prior to use

PBS

Dialysis tubing (12400 MW cut off)

Polyethylene glycol (MW 8000)

100 mM sodium hydrogen carbonate pH 9.0, this should be made up as required for dialysis

1 mM sodium periodate, prepare immediately prior to use in HPLC quality water cooled to 4° C.

Ethylene glycol

Sodium borohydride (50 mg/ml) made up immediately prior to use

All solutions prepared in HPLC quality water.

Method

Day 1—Derivatization of rTbpB

rTbpB was dialysed against 20 mM ammonium bicarbonate buffer, pH 7.0 using 3 changes of buffer and freeze dried to concentrate.

10 mg rTbpB was dissolved in 18 ml 50 mM Hepes pH 8.5.

225 μl of lysine solution was added to the rTbpB solution in a glass bottle.

2 ml of a 25 mg/ml solution of DMS was prepared and immediately 1 ml added to the protein/lysine mixture. This was done in a glass bottle because DMS reacts with metal and plastic. This solution was then mixed for 1 h at room temperature.

A further 2 ml of lysine solution was added to stop the reaction.

The solution was dialysed overnight against PBS (3 changes).

100 μl was removed for trinitrobenzenesulfonate (TNBS) assay and SDS-PAGE.

Day 2—Concentrate the Dialysed, Derivatized rTbpB to 1-2 ml with Polyethylene Glycol (PEG).

To do this the dialysed, derivative was left in the dialysis tubing and placed into a container and covered with PEG. This was left at room temperature and checked periodically until the volume had reduced to 1-2 ml. This solution was stable at 4° C. below pH 8.0.

The concentrated rTbpB derivative was dialysed against 100 mM sodium bicarbonate, pH 9.0 overnight at 4° C. (3 changes).

100 μl was removed for TNBS assay and SDS-PAGE.

Day 3—Periodate Oxidation of Meningococcal C Polysaccharide

A solution of 1 mM sodium periodate in HPLC quality water (cooled to 4° C.) was prepared.

Polysaccharide was dissolved in 1 ml of this periodate solution and mixed in the dark for 30 min at 4° C. The amount of polysaccharide used is based on molar ratio and depends on the number of moles of protein in 18 ml Hepes buffer.

1 drop of ethylene glycol was added.

Immediately, this was added to the derivatized rTbpB solution and incubated at 4° C. for 2 h.

A 50 mg/ml solution of sodium borohydride in HPLC quality water was freshly prepared and 200 μl of this solution added immediately to the reaction mixture, and incubated at 4° C. overnight.

Day 4

The rTbpB-menC conjugate was dialysed against 3 changes of PBS (prepared in HPLC quality water), to remove any unconjugated, hydrolysed polysaccharide.

The conjugate was then stored at 4° C. until required for purification by size exclusion chromatography on a Superdex 200 column (Amersham-Pharmacia).

FIG. 1 shows size exclusion chromatography of an unconjugated mixture of rTbpB and menC polysaccharide. The refractive index profile shows elution of the polysaccharide from the size exclusion column and the UV profile indicates the elution profile of the protein. The polysaccharide and rTbpB elute as separate peaks in the unconjugated mixture.

FIG. 2 shows size exclusion chromatography of conjugated rTbpB and MenC polysaccharide using the reductive amination protocol described above. The refractive index and UV profiles demonstrate coelution of protein and polysaccharide and a shift in the rTbpB protein peak towards the void volume of the size exclusion column, indicating an increase in molecular weight and confirming conjugation of the protein and polysaccharide.

### EXAMPLE 2

### Recombinant Human Transferrin Expression in E.coli

As an alternative to using human blood derived transferrin for the purification of rTbps, we have expressed a recombinant form of human transferrin in E.coli. We have expressed individual lobes of the transferrin protein, along with full length protein.

Human transferrin was cloned by PCR amplification of an existing gene clone (cDNA sequence Funmei Yang et al., (1984) PNAS 81: 2752-2756). Before use, the internal NdeI sites present in the hTf gene were removed by mutagenic PCR, as follows:

1. PCR amplification of the transferrin with the oligomers below removed the first NdeI site at amino acids 25-26, without changing the amino acid sequence. An NruI site is included in the 5′ primer, enabling the product to be cloned into a previously engineered version of hTf containing an NruI site just upstream of the NdeI site (also engineered without changing the amino acid sequence).

Primers for Removing 5′ (Upstream) NdeI Site

5′ TTT CGC GAC CAC ATG AAA AGC GTC ATT CCA TCC 3′ (5′ primer)

5′ GTT CTA GAG TGG CAG CCC TAC CTC TGA G 3′ (3′ primer)

2. Removal of the second NdeI site was a two step process: firstly, a version of hTf was generated containing an appropriately placed PvuI site in it (amino acids 642-645). The PvuI site was introduced by PCR amplification of hTf lacking the upstream NdeI site (generated as detailed above) with the following oligomers

Primers for Introducing PvuI Site into hTf

5′ CAT ATG GTC CCT GAT AAA ACT GTG AG 3′ (5′ primer)

5′ CGA TCG TGA AGT TTG GCC AAA CAT ACT G 3′ (3′ primer)

Then the 3′ end of hTf was amplified using the following oligomers:

Primers for Removing 3′ (Downstream) NdeI Site

5′ CGA TCG AAA CAC GTA TGA AAA ATA CTT AG 3′ (5′ primer)

5′ GTT CTA GAG TGG CAG CCC TAC CTC TGA G 3′ (3′ primer)

3. The PvuI sites were used to join the two products together, forming a full length recombinant hTf gene with a single NdeI site at the level of the start ATG codon.

The N terminal clone was prepared by PCR, using the oligomers below, generating an N terminus clone without the native leader sequence, encompassing amino acids 1-337 of the mature transferrin sequence.

N Terminus Clone Primers

5′ CAT ATG GTC CCT GAT AAA ACT GTG AG 3′ (5′ primer)

5′ TCT AGA TTA ATC TGT TGG GGC TTC TGG GCA TG 3′ (3′ primer)

The C terminal lobe was amplified using the oligomers below, which again enabled cloning into the NdeI site of pET and pMTL vectors, and encompassed amino acids 338-679 of the mature transferrin sequence.

C Terminus Clone Primers

5′ CAT ATG GAA TGC AAG CCT GTG AAG TGG 3′ (5′ primer)

5′ GTT CTA GAG TGG CAG CCC TAC CTC TGA G 3′ (3′ primer)

Full-length and hTf lobes were cloned into pET22b and pET26b, initially, on an NdeI-XbaI fragment.

Expression Studies

Expression studies were carried out by growing E.coli BL21 DE3 carrying the hTf pET22b and pET26b clones, to OD₆₅₀ 0.7-1.0. Expression was induced with 1 mM IPTG and hTf production monitored over the course of two hours by dot blot and Western blotting, using a goat anti-human transferrin polyclonal antibody (Sigma). The size of full length and C terminus recombinant matched that expected for unglycosylated human transferrin and its individual lobes. Microscope examination revealed that expression of hTf resulted in the production of inclusion bodies. This precipitated material requires solubilisation and refolding in order to generate functional material.

Protocol for Recombinant Transferrin Refolding and Affinity Column Generation

The protocol for solubilisation and refolding has been described elsewhere (Hoefkins P., et al. (1996) Int. J. Biochem. Cell. Biol. 28, 975-982). Briefly:

1. Isolate inclusion body material by standard cell lysis and centrifugation.

2. Dissolve pelleted protein in 8M urea, 1 mM DTT, 40 mM Tris/HCl, 10% glycerol (v/v) pH 7.6.

3. Dilute dissolved protein in renaturation buffer (0.1 mM Na-EDTA, 0.1 mM Tris/HCl, 1.0 mM reduced glutathione (GSH), pH 8.2) to a concentration of 20 μg/ml.

4. Incubate at 6° C. for 15 min.

5. Add oxidised glutathione (GSSG) to a final concentration of 0.5 mM.

6. Incubate for further 22 hr at 6° C.

7. Concentrate and dialyse against 10 mM NaHCO₃.

8. Saturate with iron and assess purity (where necessary, clean up using size exclusion chromatography or other chromatographic technique).

9. Conjugate with Sepharose 4B (Amersham Pharmacia) to generate affinity matrix.

The invention thus provides iron uptake proteins for use as carrier proteins for vaccine applications.

### EXAMPLE 3

### Methods for Assessment of TbpB-MenC Conjugate

3.1 Immunisations

To determine the immune response to conjugates, groups of five Balb/c mice (female, 6-9 weeks old) were immunised with two doses of conjugate vaccine containing 10 μg polysaccharide or unconjugated control antigens; protective efficacy was determined after groups of mice received three doses of the above preparations. All vaccines were adjuvanted with aluminium phosphate (Adjuphos, Superfos-Biosector, Frederikssund, Denmark) to give a final concentration of 4 mgml⁻¹ aluminium phosphate. Sub-cutaneous injections were performed on days 1 and 28 for immune response and on days 1, 21 and 28 for assessment of protective efficacy. Sera were collected on days 0, 14, 21 and 35 and assayed for antibodies against menCPs and TbpB by ELISA for whole IgG.

3.2 Murine IP Infection Mode for N. meningitidis Immunoassays

To determine the protective efficacy of TbpB-menCPs conjugate, Balb/c mice (female 6-8 weeks old, Harlan) were immunised on days 1, 21 and 28 with conjugate or an unconjugated mixture of polysaccharide and protein containing 10 μgml⁻¹ polysaccharide or dFim. The mice were infected on day 35 by intraperitoneal (i.p.) injection. Bacteria were grown in Mueller Hinton broth for 4 h, adjusted to the required density with the same medium, and mixed with an equal volume of sterile human transferrin (40 mgml⁻¹, Sigma) in PBS. Mice received the appropriate challenge dose i.p. in a 0.5 ml suspension amd 24 h later a second i.p. injection of 0.2 ml saline containing human transferrin (50 mgml⁻¹) was administered. Susceptibility to infection was monitored for 4 days after infection and the end-point was reached when mice exhibited symptoms of closed eyes, ruffled fur and immobility, at which time mice were euthanatised.

Referring to FIGS. 3 and 4, the TbpB-MenC conjugate provides better protection against meningococcal challenge than does TbpB alone.

3.3 ELISA

96-well microtitration plates (Immulon) were coated with 1 μg ml⁻¹ TbpB or de-0-acetylated menCPs-methylated human serum albumin (menCPs-mHSA) in PBS overnight at 4° C. and incubated with serum dilutions. Specific antibodies were detected using goat anti-mouse IgG-HRP and anti-mouse IgM and IgG isotype-HRP conjugates (Jackson) and TMBlue (Universal Biologicals). The antibody titre was defined as the reciprocal of the dilution of serum corresponding to the mid-point of the dose response curve. This was calculated using interpolation software (Genesis; Labsystems) on dose-response curves generated from eight dilutions of each serum. Interplate variation was corrected by using a pool of day 14 sera, and sera showing a titre less than the detection limit were assigned an arbitary titre of 50 for calculation of geometric means. An avidity ELISA using thiocyanate as the chaotropic agent for anti-menCPs antibodies was carried out on day 35 sera.

The immunogenicity data illustrated in FIGS. 5 and 6 shows antibodies (serum IgG) raised that recognise, separately, MenC capsule polysaccharide and TbpB antigens. In FIG. 5, the murine-produced serum IgG is challenged with menC capsular polysaccharide antigen, whereas in FIG. 6 a transferrin binding protein B antigen is employed for the IgG challenge. The anti-MenC antibodies are greatest at day 21 with the conjugate according to the present invention (see FIG. 5).

### REFERENCES

Berish, S. A., Chen, C. Y., Mietzner, T. A., and Morse, S. A. (1992): Expression of a functional neisserial fbp gene in Escherichia coli. Mol. Microbiol., 6:2607-2615.

Bouchet A, Valvano M A, Dho-Moulin M, et al. Immunological variants of the aerobactin-cloacin DF13 outer membrane protein receptor lutA among enteric bacteria. (1994) Infect Immun 62: 3017-21

Boulanger P, Ie Maire M, Bonhivers M, et al. Purification and structural and functional characterization of FhuA, a transporter of the Escherichia coli outer membrane. (1996) Biochemistry, 35: 14216-24

Boulton, I. C., Gorringe, A. R., Allison, N., Robinson, A., Gorinsky, B., Joannou, C. L., and Evans, R. W. (1998): Transferrin-binding protein B isolated from Neisseria meningitidis discriminates between apo and diferric human transferrin. Biochem. J., 334:269-273

Griffiths E. and Williams P. (1999). The iron uptake systems of pathogenic bacteria, fungi and protozoa. In Iron and Infection, Second Edition. Edited by J J Bullen and E. Griffiths. Pp 87-212. John Wiley and Sons, Chichester, UK.

Lewis L A, Gipson M, Hartman K, et al. Phase variation of HpuAB and HmbR, two distinct haemoglobin receptors of Neisseria meningitidis DNM2. (1999). Mol Microbiol 32: 977-89

Pettersson A, Maas A, van Wassenaar D, et al. Molecular characterization of FrpB, the 70-kilodalton iron-regulated outer membrane protein of Neisseria meningitidis. (1995). Infect Immun 63: 4181-4

Schryvers, A. B., Bonnah, R., Yu, R. H., Wong, H., and Retzer, M. (1998): Bacterial lactoferrin receptors. Adv. Exp. Med. Biol., 443:123-133.

Wachi M, Takada A, Nagai K. Overproduction of the outer-membrane proteins FepA and FhuE responsible for iron transport in Escherichia coli hfq::cat mutant. (1999). Biochem Biophys Res Commun, 264: 525-9.

1. A conjugate comprising:
(a) a capsular polysaccharide antigen, conjugated to
(b) a carrier, wherein the carrier is a protein associated with iron uptake by pathogenic microorganisms.

2. A conjugate according to claim 1, wherein the carrier is a bacterial iron uptake protein.

3. A conjugate according to claim 2, wherein expression of the carrier is increased or upregulated during infection.

4. A conjugate according to any of claims 1 to 3, comprising:
(a) a capsular polysaccharide antigen, conjugated to
(b) a carrier selected from
(i) TbpA,
(ii) TbpB,
(iii) a fragment of (i) or (ii),
(iv) a derivative of (i) or (ii) and
(v) mixtures of any of (i) to (iv).

5. A conjugate according to claim 4, wherein the carrier is TbpB or a fragment or derivative thereof.

6. A conjugate according to claim 5 wherein the antigen is a bacterial capsular polysaccharide.

7. A conjugate according to claim 6 comprising a pneumococcal and/or a meningococcal polysaccharide.

8. A conjugate according to any previous claim, comprising:
(a-1) a first capsular polysaccharide antigen; and
(a-2) a second antigen different from the first; both conjugated to (b) the carrier.

9. A vaccine comprising a conjugate according to any of claims 1 to 8.

10. A method of conjugating an antigen to a carrier, comprising:
(a) derivatizing an iron uptake protein; and
(b) combining the derivatized iron uptake protein from (a) with the capsular polysaccharide antigen so as to allow conjugation of the antigen to the iron uptake protein.

11. Use of a transferrin binding protein in manufacture of a carrier-antigen conjugate for vaccination.

12. A method of vaccination comprising administering an effective amount of a conjugate according to any of claims 1 to 8.

13. An affinity matrix for purification of a conjugate according to any of claims 1 to 8 comprising an immobilized ligand for an iron uptake protein.

14. A method of purifying a conjugate according to any of claims 1 to 8, comprising eluting said conjugate through an affinity matrix comprising an immobilized ligand for an iron uptake protein.
